(19) United States
(12) Patent Application Publication
(10) Pub. No.: US 2016/0206533 A1
(43) Pub. Date: Jul. 21, 2016

(54) TRANSLUCENT HAIR CONDITIONING COMPOSITION

(71) Applicant: The Procter & Gamble Company, Cincinnati, OH (US)

(72) Inventors: Cedric Kofi Aurelien CALLENS, Singapore (SG); TianYong LIM, Singapore (SG); Toshiyuki IWATA, Singapore (SG)

(21) Appl. No.: 14/995,446

(22) Filed: Jan. 14, 2016

Related U.S. Application Data
(60) Provisional application No. 62/103,570, filed on Jan. 15, 2015.

(51) Int. Cl. A61K 8/34; A61Q 5/12

(52) U.S. Cl. CPC A61K 8/345; A61Q 5/12; A61K 2800/20

(57) ABSTRACT

Disclosed is a hair conditioning composition comprising: a cationic surfactant; a high melting point fatty compound having a melting point of 25° C. or higher; a material having a refractive index of from about 1.30 to about 1.70; and an aqueous carrier, wherein a composition has a transmittance of at least about 0.5%. The composition is further specified by either: (i) the material is a polyol having at least 3 hydroxyl groups and the polyol is contained at a level of above 30% to about 80% by weight of the composition; or (ii) the cationic surfactant is a salt of a mono-long alkyl amine and an acid. The compositions provides both translucent appearance and conditioning benefits, especially conditioning benefit on wet hair when used as a rinse-off conditioner and/or spreadability on hair.

### FIELD OF THE INVENTION

The present invention relates to a hair conditioning composition comprising: a cationic surfactant; a high melting point fatty compound having a melting point of 25° C. or higher; a polyol having at least 3 hydroxyl groups; and an aqueous carrier, wherein a composition has a transmittance of at least about 0.5%, and wherein the polyol is contained at a level of above 30% to about 80% by weight of the composition.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits.

For example, Japanese Patent Application Laid-open No. H10-7532 from Kose relates to a hair conditioning composition comprising 0.1-8 wt % of a solid fatty alcohol, 0.1-5 wt % of a liquid fatty alcohol, 0.1-5 wt % of a cationic surfactant, 5-30 wt % of polyol, and 0.1-20 wt of a silicone derivative. This Kose publication also discloses a comparative example of the composition comprising 6% of cetyl alcohol, 3% of oleyl alcohol, 3% of demethylpolysiloxane, 1% of stearyltrimethylammonium chloride, and 50% of propylene glycol (Comparative example 10). This composition of Comparative example 10 is said that its feeling during application is slightly dissatisfied.

Separately, there is a need for conditioning compositions having transparent and/or translucent appearance. For example, Japanese Patent Application Laid-open No. 2001-181142 from Sakamoto Yakuhin Kogyo relates to a transparent hair care cosmetic composition comprising 3-30 wt % of a glycerin and/or polyglycerin, 1-10 wt % of a polyether modified silicone, 0.1-5 wt % of a cationic high molecular compound, and 0.1-5 wt % of a cationic surfactant. Another example is U.S. Pat. No. 5,328,685 from Helen Curtis which relates to a clear hair conditioning composition comprising: (a) about 0.4% to about 15% by weight of an amidoamine salt; (b) about 0.1% to about 5% by weight of a silicone compound having at least two quaternary ammonium moieties; and (c) an aqueous carrier. The US patent also discloses that the carrier is predominantly water but organic solvents also can be included, and discloses clear conditioning composition in examples containing 10% hexylene glycol and 4% propylene glycol, together with an amidoamine, lactic acid, and silicone. Also, there is a conditioner product example named “La Source” from Crabtree & Evelyn which contains Cetrimonium chloride, Cetyl alcohol, Glycerin, Butylene glycol, but with far lower transmittance (being from about 0.04% to about 0.06%).

Separately, H. Akatsuka etc. disclose in Col. Surf. A 326 (2008) 169-174 the effects of polyols on the rheological properties of ternary systems including behenyl trimethyl ammonium chloride (C22TAC), 1-octadecanol (C18OH) to improve the stability against shear forces, and also discloses the addition of 20-80 wt % of propylene glycol or glycerin to the system containing 2 wt % of behenyl trimethyl ammonium chloride and 4 wt % of 1-octadecanol. Transmittances of such systems with polyols are not described in this publication, however, are thought to be low in view of bilayer repeat distance shown in FIG. 8 in this publication.

However, there remains a need for such conditioning compositions to provide both translucent appearance and conditioning benefits, especially conditioning benefit on wet hair when used as a rinse-off conditioner and/or spreadability on hair.

None of the existing art provides all of the advantages and benefits of the present invention.

### SUMMARY OF THE INVENTION

The present invention is directed to a hair conditioning composition comprising:
(a) a cationic surfactant;
(b) a high melting point fatty compound having a melting point of 25° C. or higher;
(c) a polyol having at least 3 hydroxyl groups; and
(d) an aqueous carrier
wherein a composition has a transmittance of at least about 0.5%,
and wherein the polyol is contained at a level of above 30% to about 80% by weight of the composition.

The composition of the present invention provides translucent appearance and conditioning benefits, especially conditioning benefit on wet hair when used as a rinse-off conditioner and/or spreadability on hair. The present invention is also directed to a hair conditioning composition comprising:
(a) a cationic surfactant;
(b) a high melting point fatty compound having a melting point of 25° C. or higher;
(c) a polyol having at least 3 hydroxyl groups; and
(d) an aqueous carrier
wherein a composition has a transmittance of at least about 0.5%,
wherein the polyol is contained at a level of 10% to about 80% by weight of the composition,
and wherein the composition is substantially free of preservatives.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

### Hair Conditioning Composition

The hair conditioning composition of the present invention comprises a cationic surfactant, high melting point fatty compound, polyol, and an aqueous carrier. The cationic surfactant, the high melting point fatty compound, polyol, and the aqueous carrier are in the form of emulsion.

In the composition, a total amount of cationic surfactants and high melting point fatty compounds is preferably from about 1.5% to about 10%, more preferably from about 2.0% to about 8.0%, still more preferably from about 2.5% to about 6.0%, in view of providing a balance between conditioning benefits and translucent product appearance.

### Transmittance

The composition of the present invention has a translucent product appearance. The composition has a transmittance of at least about 0.5% at 25° C., preferably at least about 1.0%, more preferably at least about 1.5% and preferably up to about 40%, more preferably up to about 30%, still more preferably up to about 20%. This transmittance is for the composition, i.e., final product formulation which can contain additional ingredients such as silicone, perfume and/or preservatives.

The transmittance for the composition base before containing such additional ingredients can be higher. The composition base, thus, consist only of the cationic surfactant, the high melting point fatty compound, the polyol, and the aqueous carrier. The composition base has a transmittance of preferably at least about 5% at 25° C., more preferably at least about 7%, still more preferably at least about 10% and preferably up to about 40%, more preferably up to about 30%, still more preferably up to about 25%.

The transmittance in the present invention is a total transmittance which is a sum of diffuse and regular transmission, and which is measured at 600 nm using a Dual-beam spectrophotometer UltraScan Vis, which is a UV visible spectrophotometer available from HunterLab.

### Shear Stress

Preferably, in view of spreadability, the composition has a shear stress of at least about 100 Pa, more preferably at least about 120 Pa, still more preferably at least about 140 Pa, and preferably up to about 1000 Pa, more preferably up to about 800 Pa, still more preferably up to about 600 Pa, even more preferably up to about 400 Pa. When the shear stress is too low, the composition is too runny to spread on hair, and drip off from hair and/or hands. When the shear stress is too high, the composition is too hard to spread on hair. This shear stress is for the composition, i.e., final product formulation which can contain additional ingredients such as silicone, perfume and/or preservatives.

The shear stress for the composition base before containing such additional ingredients can be higher. The composition base, thus, consist only of the cationic surfactant, the high melting point fatty compound, the polyol, and the aqueous carrier. The composition base has a shear stress of preferably at least about 200 Pa, more preferably at least about 250 Pa, still more preferably at least about 300 Pa, even more preferably at least about 350 Pa, and preferably up to about 1000 Pa, more preferably up to about 800 Pa, still more preferably up to about 600 Pa, even more preferably up to about 400 Pa.

Shear stress is measured by shear rate sweep condition with a rheometer available from TA Instruments with a mode name of ARG2. Geometry has 40 mm diameter, 2° C. cone angle, and gap of 49 μm. Shear rate is logarithmically increased from 0 to 1200/s for 1 min, and temperature is kept at 26.7° C. Share stress at a high shear rate 950/s is measured and defined above.

### Substantially Free of Low Melting Point Fatty Compound

Preferably, the compositions of the present invention are substantially free of low melting point fatty compounds, in view of stability of the composition such as reduced the risk of phase separation, in view of spreadability by having a preferred shear stress, and/or in view of translucent product appearance

In the present invention, the compositions being “substantially free” of low melting point fatty compounds means that: the composition is free of low melting point fatty compounds; or, if the composition contains low melting point fatty compound, the level of such low melting point fatty compound is very low. In the present invention, the level of such low melting point fatty compound is, if included, 0.1% or less, preferably 0.05% or less, more preferably 0.01% or less, most preferably 0%.

The low melting point fatty compound herein have a melting point of below 25° C. (not including 25° C.), preferably below 40° C. (not including 40° C.), more preferably below 45° C. (not including 45° C.).

Such low melting point fatty compounds herein include, for example, oleyl alcohol, lauryl alcohol, isopropyl isostearate, isostearyl alcohol, 2-hexyl-1-decanol, caprylic alcohol, decyl alcohol.

### Substantially Free of Non-Silicone Thickening Polymers

Preferably, the compositions of the present invention are substantially free of non-silicone thickening polymers, in view of wet conditioning. Non-silicone thickening polymers herein are polymeric compounds other than silicone polymers, and selected from the group consisting of non-silicone nonionic thickening polymers, non-silicone cationic thickening polymers, non-silicone anionic crosslinked thickening polymers, and mixtures thereof. Non-silicone thickening polymers herein have a molecular weight of at least about 1,000.

In the present invention, the compositions being “substantially free” of non-silicone thickening polymers means that: the composition is free of non-silicone thickening polymers; or, if the composition contains non-silicone thickening polymers, the level of such non-silicone thickening polymers is very low. In the present invention, the level of such non-silicone thickening polymers is, if included, 0.1% or less, preferably 0.05% or less, more preferably 0.01% or less, most preferably 0%.

Non-silicone nonionic thickening polymers include, for example, polysaccharide polymers such as hydroxyethyl cellulose, hydroxypropyl cellulose, guars which are often used as viscosity modifiers/rheology builders. Non-silicone cationic thickening polymers include, for example, cationic celluloses such as polyquaternium-4, polyquaternium-10, cationic guars, and other non-polysaccharide cationic polymers. Non-silicone anionic crosslinked thickening polymers include, for example, carbomer, and Acrylates/C10-30 Alkyl Acrylate Crosspolymer. Substantially free of preservatives

Preferably, the compositions of the present invention are substantially free of preservatives, in view of product appearance and/or shear stress.

In the present invention, the compositions being “substantially free” of non-silicone thickening polymers means that: the composition is free of preservatives; or, if the composition contains preservatives, the level of such preservatives is very low. In the present invention, the level of such preservatives is, if included, 0.1% or less, preferably 0.05% or less, more preferably 0.01% or less, most preferably 0%.

Preservatives include, for example, parabens, aldehydes, methylchloroisothiazolinone and methylisothiazolinone, p-hydroxybenzoates, and in particular methylparaben, propylparaben, glutaraldehyde.

It is believed that a higher level of polyol such as glycerin could work as a preservative in the composition of the present invention.

### Cationic Surfactant

The compositions of the present invention comprise a cationic surfactant. The cationic surfactant can be included in the composition at a level of preferably from about 0.3%, more preferably from about 0.5%, still more preferably from about 0.7% by weight of the composition and preferably to about 5%, more preferably to about 3%, still more preferably to about 2% by weight of the composition, in view of balance between providing translucent product appearance and providing conditioning benefits

Cationic surfactant useful herein can be one cationic surfactant or a mixture of two or more cationic surfactants. Preferably, the cationic surfactant is selected from: mono-long alkyl quaternized ammonium salt; a combination of mono-long alkyl quaternized ammonium salt and di-long alkyl quaternized ammonium salt; mono-long alkyl amine; a combination of mono-long alkyl amine and di-long alkyl quaternized ammonium salt.

Among them, cationic surfactants being mono-long alkyl amines, more specifically, mono-long alkyl amidoamines are preferred in view of providing translucent product appearance and conditioning benefits.

### Mono-Long Alkyl Amine

Mono-long alkyl amine useful herein are those having one long alkyl chain of preferably from 12 to 30 carbon atoms, more preferably from 16 to 24 carbon atoms, still more preferably from 18 to 22 alkyl group. Mono-long alkyl amines useful herein also include mono-long alkyl amidoamines Primary, secondary, and tertiary fatty amines are useful.

Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethyl amine, palmitamidopropyldimethylamine, palmitamidopropyldiethyl amine, palmitamidoethyldiethylamine, palmitamidoethyldimethylamine, behenamidopropyldimethylamine, behenamidopropyldiethyl amine, behenamidoethyldiethylamine, behenamidoethyldimethylamine, arachidamidopropyldimethyl amine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al.

These amines are used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid, at a molar ratio of the amine to the acid of from about 1:0.3 to about 1:2, more preferably from about 1:0.4 to about 1:1.

### Mono-Long Alkyl Quaternized Ammonium Salt

The mono-long alkyl quaternized ammonium salts useful herein are those having one long alkyl chain which has from 12 to 30 carbon atoms, preferably from 16 to 24 carbon atoms, more preferably C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

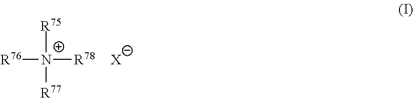

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H_{S}OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Di-Long Alkyl Quaternized Ammonium Salts

When used, di-long alkyl quaternized ammonium salts are preferably combined with a mono-long alkyl quaternized ammonium salt or mono-long alkyl amine salt, at the weight ratio of from 1:1 to 1:5, more preferably from 1:1.2 to 1:5, still more preferably from 1:1.5 to 1:4, in view of stability in rheology and conditioning benefits.

Di-long alkyl quaternized ammonium salts useful herein are those having two long alkyl chains of from 12 to 30 carbon atoms, more preferably from 16 to 24 carbon atoms, still more preferably from 18 to 22 carbon atoms. Such di-long alkyl quaternized ammonium salts useful herein are those having the formula (I):

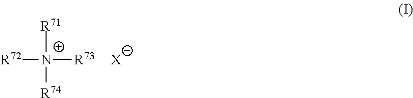

wherein two of R⁷¹, R⁷², R⁷³ and R⁷⁴ are selected from an aliphatic group of from 12 to 30 carbon atoms, preferably from 16 to 24 carbon atoms, more preferably from 18 to 22 carbon atoms or an aromatic, alkoxy, polyoxyalkylene, alkylamido, hydroxyalkyl, aryl or alkylaryl group having up to about 30 carbon atoms; the remainder of R⁷¹, R⁷², R⁷³ and R⁷⁴ are independently selected from an aliphatic group of from 1 to about 8 carbon atoms, preferably from 1 to 3 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of halides such as chloride and bromide, C1-C4 alkyl sulfate such as methosulfate and ethosulfate, and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Preferably, two of R⁷¹, R⁷², R⁷³ and R⁷⁴ are selected from an alkyl group of from 12 to 30 carbon atoms, preferably from 16 to 24 carbon atoms, more preferably from 18 to 22 carbon atoms; and the remainder of R⁷¹, R⁷², R⁷³ and R⁷⁴ are independently selected from CH₃, C₂H₅, C₂H₄OH, CH₂C₆H₅, and mixtures thereof.

Such preferred di-long alkyl cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### High-Melting Point Fatty Compound

The compositions of the present invention comprise the high melting point fatty compound. By the use of high melting point fatty compound, the composition of the present invention provides improved conditioning benefits such as friction reduction during conditioner application, ease of spreading, and/or ease of detangling, compared to compositions containing no high melting point fatty compounds and/or compared to compositions containing low melting point fatty compounds instead of high melting point fatty compounds. By the use of high melting point fatty compound, the composition of the present invention may provide improved stability of the composition such as reduced the risk of phase separation, compared to compositions containing no high melting point fatty compounds and/or compared to compositions containing low melting point fatty compounds instead of high melting point fatty compounds.

The high melting point fatty compound can be included in the composition at a level of preferably from about 1.0%, more preferably from about 1.5%, still more preferably from about 2.0%, even more preferably from about 2.5% by weight of the composition in view of providing conditioning benefits, and preferably to about 10%, more preferably to about 8.0%, still more preferably to about 5.0%, even more preferably to about 4.5% by weight of the composition, in view of providing translucent product appearance.

The high melting point fatty compound useful herein have a melting point of 25° C. or higher, preferably 40° C. or higher, more preferably 45° C. or higher, still more preferably 50° C. or higher, in view of stability of the emulsion especially the gel matrix. Preferably, such melting point is up to about 90° C., more preferably up to about 80° C., still more preferably up to about 70° C., even more preferably up to about 65° C., in view of easier manufacturing and easier emulsification. In the present invention, the high melting point fatty compound can be used as a single compound or as a blend or mixture of at least two high melting point fatty compounds. When used as such blend or mixture, the above melting point means the melting point of the blend or mixture.

The high melting point fatty compound useful herein is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than the above preferred in the present invention. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols.

Preferred fatty alcohols include, for example, cetyl alcohol (having a melting point of about 56° C.), stearyl alcohol (having a melting point of about 58-59° C.), behenyl alcohol (having a melting point of about 71° C.), and mixtures thereof. These compounds are known to have the above melting point. However, they often have lower melting points when supplied, since such supplied products are often mixtures of fatty alcohols having alkyl chain length distribution in which the main alkyl chain is cetyl, stearyl or behenyl group.

In the present invention, more preferred fatty alcohol is a mixture of cetyl alcohol and stearyl alcohol.

Generally, in the mixture, the weight ratio of cetyl alcohol to stearyl alcohol is preferably from about 1:9 to 9:1, more preferably from about 1:4 to about 4:1, still more preferably from about 1:2.3 to about 1.5:1

### Polyol

The composition comprises a polyol, which contributes to providing translucent product appearance.

In preferred embodiment, polyols can be included in the composition at a level of above 30% (not including 30%), more preferably from about 40%, still more preferably from about 45%, and preferably to about 80%, more preferably to about 70%, more preferably to about 65% by weight of the composition, in view of providing translucent appearance and having preferred shear stress. It is also preferred to have a certain weight ratio between polyol and a total weight of cationic surfactants and high melting point fatty compounds in view of providing translucent appearance. The weight ratio of polyols to the combination of cationic surfactants and high melting point fatty compounds is preferably from about 6:1 to about 18:1, more preferably from about 8:1 to about 15:1, still more preferably from about 9:1 to about 13:1.

Polyol useful herein are those having a molecular weight of preferably from about 60 to about 500, more preferably from about 60 to about 350, still more preferably from about 60 to about 200, even more preferably from about 60 to about 150.

Polyols useful herein have at least 3 hydroxyl groups, and preferably up to 12 hydroxyls groups, more preferably up to 10 hydroxyls groups, still more preferably up to 6 hydroxyls groups, even more preferably up to 4 hydroxyls groups.

Polyols useful herein are preferably water soluble. Water soluble polyols herein means those being soluble at a level used at 30° C. Non-water soluble polyols are, for example, glyceryl stearate that requires higher temperature to be soluble. Preferably, polyols herein are free of ester bond and ether bond.

Among such polyols useful herein, preferred are glycerin, sorbitol, and xylitol, in view of providing translucent appearance and preferred shear stress. More preferred are glycerin, in view of translucent appearance and preferred shear stress.

### Aqueous Carrier

The composition of the present invention preferably comprises an aqueous carrier. The level and species of the carrier are selected according to the compatibility with other components, and other desired characteristic of the product.

The carrier useful in the present invention includes water and water solutions of lower alkyl alcohols. The lower alkyl alcohols useful herein are monohydric alcohols having 1 to 6 carbons, more preferably ethanol and isopropanol.

Preferably, the aqueous carrier is substantially water. Deionized water is preferably used. Water from natural sources including mineral cations can also be used, depending on the desired characteristic of the product. Generally, the compositions of the present invention comprise from about 20% to about 80%, preferably from about 20% to about 70%, and more preferably from about 30% to about 60%, and more preferably from about 35% to about 55% water by weight of the composition.

### Gel Matrix

Preferably, in the present invention, the cationic surfactant system, the high melting point fatty compound, the polyol and an aqueous carrier form a gel matrix. The gel matrix is suitable for providing various conditioning benefits, such as slippery feel during the application to wet hair and softness and moisturized feel on dry hair.

Preferably, when the gel matrix is formed, the cationic surfactant and the high melting point fatty compound are contained at a level such that the weight ratio of the cationic surfactant to the high melting point fatty compound is in the range of, preferably from about 1:1 to about 1:7, more preferably from about 1:1.15 to about 1:5, still more preferably from about 1:2 to 1:4, in view of providing conditioning benefits and translucent product appearance. In this weight ratio, the cationic surfactant is a component without counter ion or acid, for example, when the cationic surfactant is the combination of a mono-long alkyl amine and an acid, the weight of mono-long alkyl amine without the weight of acid is considered in this weight ratio.

Preferably, when the gel matrix is formed, the composition of the present invention is substantially free of anionic surfactants in view of stability of the gel matrix.

In the present invention, “the composition being substantially free of anionic surfactants” means that: the composition is free of anionic surfactants; or, if the composition contains anionic surfactants, the level of such anionic surfactants is very low. In the present invention, a total level of such anionic surfactants n, if included, preferably 0.1% or less, more preferably 0.05% or less, still more preferably 0.01% or less by weight of the composition. Most preferably, the total level of such anionic surfactants is 0% by weight of the composition.

### Silicone Compound

The compositions of the present invention may contain a silicone compound. The silicone compounds are included at levels by weight of the composition of from about 0.05% to about 15%, preferably from about 0.1% to about 10%, more preferably from about 0.1% to about 8%.

Preferably, the silicone compounds have an average particle size of from about lmicrons to about 50 microns, in the composition.

The silicone compounds useful herein, as a single compound, as a blend or mixture of at least two silicone compounds, or as a blend or mixture of at least one silicone compound and at least one solvent, have a viscosity of preferably from about 1,000 to about 2,000,000 mPa·s at 25° C.

The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

In some embodiments, amino substituted silicones are preferably used. Preferred aminosilicones include, for example, those which conform to the general formula (I):

(R₁)ₐG₃ₐ-Si—(—OSiG₂)ₙ-(—OSiG_{b}(R₁)_{2-b})ₘ-O—SiG₃₋ₐ(R₁)ₐ

wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A ; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion.

Highly preferred amino silicones are those corresponding to formula (I) wherein m=0, a=1, q=3, G=methyl, n is preferably from about 1500 to about 1700, more preferably about 1600; and L is —N(CH₃)₂ or —NH₂, more preferably —NH₂. Another highly preferred amino silicones are those corresponding to formula (I) wherein m=0, a=1, q=3, G=methyl, n is preferably from about 400 to about 600, more preferably about 500; and L is —N(CH₃)₂ or —NH₂, more preferably —NH₂. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s. Other suitable alkylamino substituted silicone compounds include those having alkylamino substitutions as pendant groups of a silicone backbone. Highly preferred are those known as “amodimethicone”. Commercially available amodimethicones useful herein include, for example, BY16-872 available from Dow Corning.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; coloring agents, such as any of the FD&C or D&C dyes; perfumes; ultraviolet and infrared screening and absorbing agents such as benzophenones; and antidandruff agents such as zinc pyrithione.

### Product Forms

The compositions of the present invention can be in the form of rinse-off products or leave-on products, and can be formulated in a wide variety of product forms, including but not limited to creams, gels, emulsions, mousses and sprays. The composition of the present invention is especially suitable for hair conditioners especially rinse-off hair conditioners.

When used as a rinse-off conditioner, the composition is preferably used by the following steps:
(i) after shampooing hair, applying to the hair an effective amount of the conditioning composition for conditioning the hair; and
(ii) then rinsing the hair.

Effective amount herein is, for example, from about 0.1 ml to about 2 ml per 10 g of hair, preferably from about 0.2 ml to about 1.5 ml per 10 g of hair.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

TABLE 1
Compositions (wt %)

| Components | Ex. 1 | Ex. 2 | Ex. 3 | Ex. 4 | Ex. 5 |
|---|---|---|---|---|---|
| Stearamidopropyl dimethyl amine | 1.13 | 1.13 | 1.13 | 1.13 | 1.13 |
| L-glutamic acid | 0.37 | 0.37 | 0.37 | 0.37 | 0.37 |
| Cetyl alcohol | 1.17 | 1.17 | 1.17 | 1.17 | 1.17 |
| Stearyl alcohol | 2.0 | 2.0 | 2.0 | 2.0 | 2.0 |
| Glycerin | 50 | 60 | — | — | 50 |
| Xylitol | — | — | 50 | — | — |
| Sorbitol | — | — | — | 50 | — |
| Silicone | 0.35 | 0.35 | 0.35 | 0.35 | 0.35 |
| Preservatives | 0.9 | 0.9 | 0.9 | 0.9 | — |
| Perfume | 0.4 | 0.4 | 0.4 | 0.4 | 0.4 |
| Deionized Water | q.s. to 100% | | | | |
| % T for the composition base | 16.1 | 20.6 | 6.68 | 9.24 | 9.61 |
| % T for the composition (final | 6.09 | 2.05 | 4.65 | 3.09 | 5.04 |
| product formulation) | | | | | |
| Shear stress for the composition base | 498 | 618 | 353 | 398 | 437 |
| Shear stress for the composition | 282 | 465 | 332 | 268 | 264 |
| (final product formulation) | | | | | |
| Phase separation | ◯ | ◯ | ◯ | ◯ | ◯ |
| Spreadability on wet hair | Control | No | No | No | No |
| | | noticeable | noticeable | noticeable | noticeable |
| | | difference | difference | difference | difference |
| | | from | from | from | from |
| | | Control | Control | Control | Control |
| Wet detangling before and after | Control | No | No | No | No |
| rinsing | | noticeable | noticeable | noticeable | noticeable |
| | | difference | difference | difference | difference |
| | | from | from | from | from |
| | | Control | Control | Control | Control |

TABLE 2
Compositions (wt %)

| | CEx. | CEx. | CEx. | CEx. | CEx. | CEx. |
|---|---|---|---|---|---|---|
| Components | i | ii | iii | iv | v | vi |
| Stearamidopropyl dimethyl | 1.13 | 1.13 | 1.13 | 1.13 | 1.13 | 1.13 |
| amine | | | | | | |
| L-glutamic acid | 0.37 | 0.37 | 0.37 | 0.37 | 0.37 | 0.37 |
| Cetyl alcohol | — | 1.17 | 1.17 | 1.17 | 1.17 | — |
| Stearyl alcohol | — | 2.0 | 2.0 | 2.0 | 2.0 | — |
| Lauryl alcohol | — | — | — | — | — | 2.15 |
| Glycerin | 50 | 10 | — | — | — | 50 |
| 1,2-Propylene glycol | — | — | 50 | — | — | — |
| 1,3 Butylene glycol | — | — | — | 50 | — | — |
| PEG | — | — | — | — | 50 | — |
| Silicone | 0.35 | 0.35 | 0.35 | 0.35 | 0.35 | 0.35 |
| Preservatives | 0.9 | 0.9 | 0.9 | 0.9 | 0.9 | 0.9 |
| Perfume | 0.4 | 0.4 | 0.4 | 0.4 | 0.4 | 0.4 |
| Deionized Water | q.s. to 100% | | | | | |
| % T for the composition base | N/A | 2.17 | 2.52 | 1.70 | 0.13 | 7.05 |
| % T for the composition | N/A | 1.23 | 0.58 | 0.61 | 0.16 | N/A |
| (final product formulation) | | | | | | |
| Shear stress for the | N/A | 174 | N/A | N/A | N/A | 47 |
| composition base | | | | | | |
| Shear stress for the | N/A | 131 | N/A | N/A | N/A | N/A |
| composition (final | | | | | | |
| product formulation) | | | | | | |
| Phase separation | X | ◯ | ◯ | ◯ | ◯ | X |
| Spreadability on wet hair | N/A | N/A | N/A | N/A | N/A | N/A |
| Wet detangling before and | N/A | N/A | N/A | N/A | N/A | N/A |
| after rinsing | | | | | | |

### Method of Preparation

The hair care compositions of “Ex. 1” through “Ex.5”, “CEx.i” and “CEx.vi” as shown above can be prepared by any conventional method well known in the art.

### Properties and Conditioning Benefits

For some of the above compositions, properties and conditioning benefits are evaluated by the following methods. Results of the evaluation are also shown above.

The embodiments disclosed and represented by “Ex. 1” through “Ex. 5” are hair conditioning compositions of the present invention which are particularly useful for rinse-off use. Such embodiments have many advantages. For example, they provide translucent product appearance, conditioning benefits especially wet conditioning benefits, and also improved stability of the composition such as reduced the risk of phase separation. Such advantages of the present invention can be understood by the comparison between these examples and comparatives examples “CEx.i” and “CEx.vi”.

### Properties Such as % T and Shear Stress

% T and shear stress are measured by the methods described above.

### Phase Separation

The compositions are judged if a phase separation happens or not by direct visual observation.

◯: No phase separation is observed

×: Phase separation is observed.

### Panelist Test for Conditioning Benefits

Conditioning benefits such as spreadability and wet detangling are evaluated by a panellist test. 8 panelists evaluated samples prepared by applying 0.1 ml of the above compositions per 1 g hair. Panelists evaluated each sample from 0 to 100 scale for each of spreadability and detangling. The data from the panelists were gathered, averaged, and scored, and compared.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair conditioning composition comprising:
(a) a cationic surfactant;
(b) a high melting point fatty compound having a melting point of 25° C. or higher;
(c) a polyol having at least 3 hydroxyl groups; and
(d) an aqueous carrier
wherein a composition has a transmittance of at least about 0.5%,
and wherein the polyol is contained at a level of above 30% to about 80% by weight of the composition.

2. The composition of claim 1, wherein the polyol is glycerin.

3. The composition of claim 1 or 2, wherein the polyol is contained at a level of from about 40% to about 70%.

4. The composition of any of claims 1-3, wherein the polyol is contained at a level of from about 45% to about 65%.

5. The composition of any of claims 1-4, wherein the composition has a transmittance of at least about 1.0%.

6. The composition of any of claims 1-5, wherein the composition has a transmittance of at least about 1.5%.

7. The composition of any of claims 1-6, wherein the composition comprises a composition base,
wherein the composition base consisting of the cationic surfactant, the high melting point fatty compound, the polyol, and the aqueous carrier,
wherein the composition base has a transmittance of at least about 5%.

8. The composition of claim 7, wherein the composition base has a transmittance of at least about 7%.

9. The composition of claim 8, wherein the composition base has a transmittance of at least about 10%.

10. The composition of any of claims 1-9, wherein the composition has a shear stress of at least about 100.

11. The composition of any of claims 1-10, wherein the composition has a shear stress of at least about 120.

12. The composition of any of claims 1-11, wherein the composition has a shear stress of at least about 140.

13. The composition of any of claims 1-12, wherein the composition comprises a composition base,
wherein the composition base consisting of the cationic surfactant, the high melting point fatty compound, the polyol and the aqueous carrier,
wherein the composition base has a shear stress of at least about 200.

14. The composition of claim 13, wherein the composition base has a shear stress of at least about 250.

15. The composition of claim 14, wherein the composition base has a shear stress of at least about 300.

16. The composition of claim 15, wherein the composition base has a shear stress of at least about 350.

17. The composition of any of claims 1-16, wherein the composition is free of non-silicone thickening polymer having a molecular weight of from about 1000.

18. The composition of any of claims 1-17, wherein the composition is substantially free of a low melting point fatty compound having a melting point of below 25° C.

19. A hair conditioning composition comprising:
(a) a cationic surfactant;
(b) a high melting point fatty compound having a melting point of 25° C. or higher;
(c) a polyol having at least 3 hydroxyl groups; and
(d) an aqueous carrier
wherein a composition has a transmittance of at least about 0.5%,
wherein the polyol is contained at a level of 10% to about 80% by weight of the composition, and wherein the composition is substantially free of preservatives.
